(19) United States
(12) Patent Application Publication
(10) Pub. No.: US 2019/0064172 A1
(43) Pub. Date: Feb. 28, 2019

(54) PROGNOSIS OF SEROUS OVARIAN CANCER USING BIOMARKERS

(71) Applicant: EISAI INC., Woodcliff Lake, NJ (US)

(72) Inventor: Daniel John O'SHANNESSY, Schwenksville, PA (US)

(21) Appl. No.: 16/093,180

(22) PCT Filed: Apr. 20, 2017
(86) PCT No.: PCT/IB2017/052289

§ 371 Date: Oct. 12, 2018

Related U.S. Application Data
(60) Provisional application No. 62/324,920, filed on Apr. 20, 2016.

(51) Int. Cl. G01N 33/574; G06F 19/12

(52) U.S. Cl. CPC G01N 33/57449; G06F 19/12; G01N 2333/575; G01N 2333/5421; G01N 2333/95; G01N 2333/71; G01N 2333/4704

(57) ABSTRACT

Described herein are methods of using biomarker levels to detect proteins in a biological sample obtained from a patient with ovarian cancer, calculate a quantitative score for a patient with ovarian cancer, and predict a likelihood of a clinical outcome in a patient with ovarian cancer. The methods involve determining a level of at least three proteins in the biological sample obtained from the patient wherein the at least three proteins are selected from ANG-2, HE4, PROSTASIN, EGFR and IL-8, calculating a quantitative score for the patient by weighting the level of the at least three proteins by their contribution to a clinical outcome, and/or predicting a likelihood of a clinical outcome for the patient based on the quantitative score. Also provided are sets of reagents and test kits to the levels of the biomarkers described herein.

### CROSS REFERENCE TO RELATED APPLICATIONS

This application claims the benefit of U.S. Provisional Patent Application No. 62/324,920, filed Apr. 20, 2016, the entirety of which is incorporated herein by reference.

### TECHNICAL FIELD

The subject matter provided herein relates to methods of detecting proteins in a biological sample obtained from a patient with ovarian cancer, methods of calculating a quantitative score for a patient with ovarian cancer, and methods of predicting a likelihood of a clinical outcome in a patient with ovarian cancer. Also provided are sets of reagents and test kits to measure biomarker levels.

### BACKGROUND

Epithelial ovarian cancer (EOC) is the leading cause of death due to gynecologic malignancy, in part because of the late stage of diagnosis in most cases and the high rate of recurrence. To date, no targeted therapeutic has been approved for use in EOC, and greater than 14,000 deaths from ovarian cancer were estimated to occur in the United States in 2014. Only modest progress on patient survival or prognosis has occurred since the introduction of platinum-based therapies for EOC nearly 30 years ago. Vaughan et al., Nature Reviews Cancer, 11, 719-725 (2011). As such, significant effort has been expended in the search for prognostic markers in EOC.

EOC has recently been shown to derive from tubal epithelia and has been redefined, molecularly, as low grade and high grade subtypes. The most prevalent EOC is high grade serous ovarian cancer (HGSOC), accounting for approximately 80% of EOC. HGSOC demonstrates a high rate of mutation in tumor protein p53. Although work continues to molecularly define EOC, and in particular HGSOC, ovarian cancer remains a heterogeneous disease with distinctly different outcomes. There is currently a need for a non-invasive, serum-based model that will allow physicians to stratify their patients for subsequent therapeutic interventions resulting in a more personalized approach to care.

### SUMMARY

Provided herein are methods for detecting proteins in a biological sample obtained from a patient with ovarian cancer. These methods comprise determining a level of at least three proteins in a biological sample obtained from the patient, wherein the at least three proteins are selected from ANG-2, HE4, PROSTASIN, EGFR and IL8.

In some embodiments, the ovarian cancer is a non-mucinous epithelial ovarian cancer. In some embodiments, the biological sample is serum, plasma, or ascites. In some embodiments the level of at least three proteins is determined using an immunoassay. In some embodiments, the immunoassay is an electrochemiluminescent assay. In some embodiments, the levels of EGFR, HE4 and IL8 are determined. In other embodiments, the levels of ANG-2, HE4, PROSTASIN, EGFR and IL8 are determined.

Also provided herein are methods for calculating a quantitative score for a patient with ovarian cancer. These methods comprise determining a level of at least three proteins in a biological sample obtained from the patient, wherein said determining step comprises determining the level of ANG-2, HE4, PROSTASIN, EGFR and IL8 and calculating a quantitative score for the patient by weighting the level of the at least three proteins by their contribution to a clinical outcome.

In some embodiments, the ovarian cancer is a non-mucinous epithelial ovarian cancer. In some embodiments, the biological sample is serum, plasma, or ascites. In some embodiments the level of at least three proteins is determined using an immunoassay. In some embodiments, the immunoassay is an electrochemiluminescent assay.

In some embodiments, the levels of ANG-2, HE4, PROSTASIN, EGFR and IL8 are determined. In some embodiments, the quantitative score is calculated based on the algorithm:

h_{OS}(t)=h0_{OS}(t) exp(˜A*ANG2+˜B*HE4+˜C*PROSTASIN−˜D*EGFR+˜E*IL8)

wherein h_{OS}(t) is the hazard at time (t) and h0_{OS}(t) is the baseline hazard with overall survival as the outcome, wherein the gene symbols in the equation represent the protein levels, and wherein coefficients A, B, C, D, and E are the coefficients derived for each respective protein, the model being optimized to provide maximal prognostic information for the given population of ovarian cancer patients. In some embodiments, the quantitative score is calculated based on the algorithm:

h_{OS}(t)=h0_{OS}(t) exp(1.213 ANG2+0.171 HE4+0.102 PROSTASIN−1.406 EGFR+0.207 IL8).

It is understood to those of skill in the art that the coefficients of the quantitative score equations provided herein are subject to some variability depending upon the population of subjects diagnosed with ovarian cancer.

In other embodiments, the quantitative score is calculated based on the algorithm:

h_{PFS}(t)=h0_{PFS}(t) exp(˜A*ANG2+˜B*HE4+˜C*PROSTASIN−˜D*EGFR+˜E*IL8)

wherein h_{PFS}(t) is the hazard at time (t) and h0_{PFS}(t) is the baseline hazard with progression free survival as the outcome, wherein the gene symbols in the equation represent the protein levels, and wherein coefficients A, B, C, D, and E are the coefficients derived for each respective protein, the model being optimized to provide maximal prognostic information for the given population of ovarian cancer patients. In some embodiments, the quantitative score is calculated based on the algorithm:

h_{PFS}(t)=h0_{PFS}(t) exp(0.077 ANG2+0.123 HE4+0.008 PROSTASIN−0.545 EGFR+0.156 IL8).

In some embodiments, the levels of EGFR, HE4 and IL8 are determined. In some embodiments, the quantitative score is calculated based on the algorithm:

h_{OS}(t)=h0_{OS}(t) exp(˜A*HE4−˜B*EGFR+˜C*IL8)

wherein h_{OS}(t) is the hazard at time (t) and h0_{OS}(t) is the baseline hazard with overall survival as the outcome, wherein the gene symbols in the equation represent the protein levels, and wherein coefficients A, B and C are the coefficients derived for each respective protein, the model being optimized to provide maximal prognostic information for the given population of ovarian cancer patients. In some embodiments, the quantitative score is calculated based on the algorithm:

h_{OS}(t)=h0_{OS}(t) exp(0.234 HE4−1.464 EGFR+0.273 IL8)

It is understood to those of skill in the art that the coefficients of the quantitative score equations provided herein are subject to some variability depending upon the population of subjects diagnosed with ovarian cancer.

In other embodiments, the quantitative score with progression-free survival as the outcome is based on the algorithm:

h_{PFS}(t)=h0_{PFS}(t) exp(˜A*HE4−˜B*EGFR+˜C*IL8)

wherein h_{PFS}(t) is the hazard at time (t) and h0_{PFS}(t) is the baseline hazard with overall survival as the outcome, wherein the gene symbols in the equation represent the protein levels, and wherein coefficients A, B, and C are the coefficients derived for each respective protein, the model being optimized to provide maximal prognostic information for the given population of ovarian cancer patients. In some embodiments, the quantitative score is calculated based on the algorithm:

h_{PFS}(t)=h0_{PFS}(t) exp(0.124 HE4−0.538 EGFR+0.161 IL8).

Also provided herein are methods for predicting a likelihood of a clinical outcome in a patient with ovarian cancer. These methods comprise determining a level of at least three proteins in a biological sample obtained from the patient, wherein the at least three proteins are selected from ANG-2, HE4, PROSTASIN, EGFR and IL8, calculating a quantitative score for the patient by weighting the level of the at least three proteins by their contribution to a clinical outcome, and predicting a likelihood of a clinical outcome for the patient based on the quantitative score.

In some embodiments, an increase in the quantitative score correlates with a decreased likelihood of a positive clinical outcome, and a decrease in the quantitative score correlates with an increased likelihood of a positive clinical outcome. In some embodiments, a likelihood of a negative clinical outcome for the patient informs a decision to discontinue current ovarian cancer therapy and/or initiate an ovarian cancer therapy, and a likelihood of a positive clinical outcome for the patient informs a decision to monitor the progression of the ovarian cancer and/or continue current ovarian cancer therapy. In some embodiments, the positive clinical outcome is increased overall survival time. In some embodiments, the positive clinical outcome is progression free survival. In some embodiments, the ovarian cancer is a non-mucinous epithelial ovarian cancer.

In some embodiments, the likelihood of a clinical outcome is predicted when the ovarian cancer is first diagnosed. In other embodiments, the likelihood of a clinical outcome is predicted when the ovarian cancer relapses for the first time 6 to 24 months after an initial treatment. In further embodiments, the likelihood of a clinical outcome is predicted when the ovarian cancer relapses at any time after an initial treatment. In still further embodiments, the likelihood of a clinical outcome is predicted at any time after a first diagnosis. In some embodiments, the initial treatment comprises surgery and/or chemotherapy. In some embodiments, the biological sample is serum, plasma, or ascites. Also disclosed are methods of predicting a likelihood of a clinical outcome in a patient with ovarian cancer, wherein the level of at least three proteins is determined using an immunoassay.

In some embodiments, the levels of ANG-2, HE4, PROSTASIN, EGFR and IL8 are determined. In some embodiments, the quantitative score is calculated based on the algorithm:

h_{OS}(t)=h0_{OS}(t) exp(˜A*ANG2+˜B*HE4+˜C*PROSTASIN−˜D*EGFR+˜E*IL8)

wherein h_{OS}(t) is the hazard at time (t) and h0_{OS}(t) is the baseline hazard with overall survival as the outcome, wherein the gene symbols in the equation represent the protein levels, and wherein coefficients A, B, C, D, and E are the coefficients derived for each respective protein, the model being optimized to provide maximal prognostic information for the given population of ovarian cancer patients. In some embodiments, the quantitative score is calculated based on the algorithm:

h_{OS}(t)=h0_{OS}(t) exp(1.213 ANG2+0.171 HE4+0.102 PROSTASIN−1.406 EGFR+0.207 IL8).

It is understood to those of skill in the art that the coefficients of the quantitative score equations provided herein are subject to some variability depending upon the population of subjects diagnosed with ovarian cancer.

In other embodiments, the quantitative score is calculated based on the algorithm:

h_{PFS}(t)=h0_{PFS}(t) exp(˜A*ANG2+˜B*HE4+˜C*PROSTASIN−˜D*EGFR+˜E*IL8)

wherein h_{PFS}(t) is the hazard at time (t) and h0_{PFS}(t) is the baseline hazard with progression free survival as the outcome, wherein the gene symbols in the equation represent the protein levels, and wherein coefficients A, B, C, D, and E are the coefficients derived for each respective protein, the model being optimized to provide maximal prognostic information for the given population of ovarian cancer patients. In some embodiments, the quantitative score is calculated based on the algorithm:

h_{PFS}(t)=h0_{PFS}(t) exp(0.077 ANG2+0.123 HE4+0.008 PROSTASIN−0.545 EGFR+0.156 IL8).

In some embodiments, the levels of EGFR, HE4 and IL8 are determined. In some embodiments, the quantitative score is calculated based on the algorithm:

h_{OS}(t)=h0_{OS}(t) exp(˜A*HE4−˜B*EGFR+˜C*IL8)

wherein h_{OS}(t) is the hazard at time (t) and h0_{OS}(t) is the baseline hazard with overall survival as the outcome, wherein the gene symbols in the equation represent the protein levels, and wherein coefficients A, B, and C are the coefficients derived for each respective protein, the model being optimized to provide maximal prognostic information for the given population of ovarian cancer patients. In some embodiments, the quantitative score is calculated based on the algorithm:

h_{OS}(t)=h0_{OS}(t) exp(0.234 HE4−1.464 EGFR+0.273 IL8)

It is understood to those of skill in the art that the coefficients of the quantitative score equations provided herein are subject to some variability depending upon the population of subjects diagnosed with ovarian cancer.

In other embodiments, the quantitative score with progression-free survival as the outcome is based on the algorithm:

h_{PFS}(t)=h0_{PFS}(t) exp(˜A*HE4−˜B*EGFR+˜C*IL8)

wherein h_{PFS}(t) is the hazard at time (t) and h0_{PFS}(t) is the baseline hazard with overall survival as the outcome, wherein the gene symbols in the equation represent the protein levels, and wherein coefficients A, B, and C are the coefficients derived for each respective protein, the model being optimized to provide maximal prognostic information for the given population of ovarian cancer patients. In some embodiments, the quantitative score is calculated based on the algorithm:

h_{PFS}(t)=h0_{PFS}(t) exp(0.124 HE4−0.538 EGFR+0.161 IL8).

Also provided are sets of reagents to measure the levels of two or more biomarkers in a patient with ovarian cancer, wherein the biomarkers comprise ANG2, EGFR, HE4, IL8 and PROSTASIN and their measurable fragments. In some embodiments, the reagents are binding molecules. In some embodiments, the binding molecules are antibodies.

Also provided are test kits comprising sets of reagents to measure the levels of two or more biomarkers in a patient with ovarian cancer, wherein the biomarkers comprise ANG2, EGFR, HE4, IL8 and PROSTASIN and their measurable fragments. In some embodiments, the test kits further comprise written instructions for performing an evaluation of biomarkers to predict the likelihood of ovarian cancer in a subject.

### BRIEF DESCRIPTION OF THE DRAWINGS

FIG. 1 provides a graphical representation of the subject disposition. Pre-treatment baseline serum samples were taken from 529 subjects, and 403 of the 529 subjects were placed in the serous sub-group. The serous sub-group subjects were analyzed in two cohorts with 132 subjects from the placebo group (the training set) and 271 subjects from the farletuzumab-treated group (the validation set).

FIG. 2A-FIG. 2E provide Kaplan-Meier (KM) plots for the 5 most significant analytes on univariate analysis for overall survival (OS) on the training cohort. FIG. 2A is the KM plot for ANG-2, FIG. 2B is the KM plot for EGFR, FIG. 2C is the KM plot for HE4, and FIG. 2D is the KM plot for IL8, and FIG. 2E is the KM plot for PROSTASIN.

FIG. 3A shows a graphical representation of lasso variable selection based on overall survival (OS) as outcome measure on the training cohort. FIG. 3B shows a graphical representation of lasso variable selection based on progression-free survival (PFS) as outcome measure on the training cohort.

FIG. 4A-FIG. 4D provide Kaplan-Meier plots for the PFS-derived (PROFILE-Ov) and OS-derived prognostic models on the training cohort. FIG. 4A is the KM plot for the PFS-derived model with PFS as the outcome and FIG. 4B is the KM plot for the PFS-derived model with OS as the outcome. FIG. 4C is the KM plot for the PFS-derived model with PFS as the outcome and FIG. 4D is the KM plot for the PFS-derived model with OS as the outcome.

FIG. 5A and FIG. 5B provide Kaplan-Meier plots for the PFS-derived prognostic model (PROFILE-Ov) on the validation cohort. FIG. 5A is the KM plot for the PFS-derived model with PFS as the outcome and FIG. 5B is the KM plot for the PFS-derived model with OS as the outcome.

FIG. 6A and FIG. 6B show PROFILE-Ov Score plots with the PROFILE-Ov Score on the x-axis and percent mortality on the y-axis for PFS (FIG. 6A) and OS (FIG. 6B).

### DETAILED DESCRIPTION OF ILLUSTRATIVE EMBODIMENTS

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a biological sample” includes a combination of two or more biological samples, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain error necessarily resulting from the standard deviation found in its respective testing measurement.

The term “comprising” is intended to include examples encompassed by the terms “consisting essentially of” and “consisting of.” The term “consisting essentially of” is intended to include examples encompassed by the term “consisting of.”

The term “patient” or “subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

As used herein, the term “ovarian cancer” is used in the broadest sense and refers to all stages and forms of cancer arising from the tissues of the ovaries. Ovarian tumors may be epithelial cell tumors, germ cell tumors, or stromal cell tumors. Epithelial ovarian cancer may be histologically categorized as serous, endometrioid, clear cell, mucinous, Brenner, transitional cell, small cell, mixed mesodermal or undifferentiated. Serous tumors may be further sub-categorized as serous cystadenoma, borderline serous tumor, serous cystadenocarcinoma, adenofibroma or cystadenofibroa. Mucinous tumors may be further sub-categorized into mucinous cystadenoma, borderline mucinous tumor, mucinous cystadenocarcinoma or adenofibroma. “Non-mucinous epithelial ovarian cancer” refers to epithelial ovarian cancers that are not histologically categorized as mucinous.

Staging of the ovarian cancer is useful for assessing disease progression and for planning treatment. According to the Jan. 1, 2014 guidelines release by the Federation Internationale de Gynecologie et d'Obstetrique (FIGO) and approved by the American Joint Committee on Cancer and the International Union Against Cancer, stage I ovarian cancer is confined to the ovaries; stage II ovarian cancer involves one or both of the ovaries with pelvic extension (below the pelvic brim) or primary peritoneal cancer, stage III ovarian cancer involves one or both of the ovaries with cytologically or histologically confirmed spread to the peritoneum outside the pelvis and/or metastasis to the retroperitoneal lymph nodes, and stage IV ovarian cancer involves distant metastasis excluding peritoneal metastasis.

“Protein,” “polypeptide” and “peptide” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. Polypeptides of the invention include conservatively modified variants. One of skill will recognize that substitutions, deletions or additions to a nucleic acid, peptide, polypeptide, or protein sequence which alter, add or delete a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Conservative substitution tables providing functionally similar amino acids are well known in the art. Such conservatively modified variants are in addition to and do not exclude polymorphic variants, interspecies homologs, and alleles of the invention.

Proteins of the invention further encompass all naturally occurring post-transcriptional and post-translational modifications to the polymer of amino acid residues. Proteins of the invention additionally encompass all chemically, enzymatically and/or metabolically modified forms of unmodified proteins. The protein may be located in the cytoplasm of the cell, or into the extracellular milieu such as the growth medium of a cell culture. The protein may be soluble or insoluble. In preferred embodiments, the protein is soluble.

The term “biological sample” as used herein refers to a collection of similar fluids, cells, or tissues (e.g., surgically resected tumor tissue, biopsies, including fine needle aspiration), isolated from a subject, as well as fluids, cells, or tissues present within a subject. In some embodiments the sample is a biological fluid. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, saliva, cystic fluid, tear drops, feces, sputum, mucosal secretions of the secretory tissues and organs, vaginal secretions, ascites such as those associated with non-solid tumors, fluids of the pleural, pericardial, peritoneal, abdominal and other body cavities, fluids collected by bronchial lavage and the like. In preferred embodiments, the biological sample is serum, plasma or ascites.

Biological fluids may also include liquid solutions contacted with a subject or biological source, for example, cell and organ culture medium including cell or organ conditioned medium, lavage fluids and the like. The term “biological sample,” as used herein, encompasses materials removed from a subject or materials present in a subject.

As describe herein, “immunoassay” can include, for example, western blot analysis, radioimmunoassay, immunofluorimetry, immunoprecipitation, immunodiffusion, electrochemiluminescence (ECL) immunoassay, immunohistochemistry, fluorescence-activated cell sorting (FACS) or ELISA assay. Such assays typically rely on one or more antibodies, for example, anti-ANG-2 antibodies. In preferred embodiments, the immunoassay is an ECL assay.

As used herein, the term “antibody” is used in its broadest sense to include polyclonal and monoclonal antibodies, as well as polypeptide fragments of antibodies that retain binding activity for the biomarkers described in this application. One skilled in the art understands that antibody fragments including Fab, F(ab′)2 and Fv fragments can retain binding activity for the biomarkers described in this application and, thus, are included within the definition of the term antibody as used herein. Methods of preparing monoclonal and polyclonal antibodies are routine in the art.

Antibodies suitable for use in the method of the invention, include, for example, monoclonal or polyclonal antibodies, fully human antibodies, human antibody homologs, humanized antibody homologs, chimeric antibodies, singles chain antibodies, chimeric antibody homologs, and monomers or dimers of antibody heavy or light chains or mixtures thereof The antibodies of the invention may include intact immunoglobulins of any isotype including types IgA, IgG, IgE, IgD, IgM (as well as subtypes thereof). The light chains of the immunoglobulin may be kappa or lambda.

As used herein, a “quantitative score” is a mathematically calculated numerical value representing the hazard at time (t), or the instantaneous rate of occurrence of an event. In some embodiments, a quantitative score may be calculated using an algorithm derived using progression-free survival as the outcome (“PROFILE-Ov”), and in other embodiments a quantitative score may be calculated using an algorithm derived using overall survival as the outcome. In some embodiments, a quantitative score may be calculated using an algorithm with overall survival as the outcome, and in some embodiments a quantitative score may be calculated using an algorithm with progression free survival as the outcome.

“Overall survival (OS),” as used in the context of ovarian cancer, refers to the length of time from either the date of diagnosis or the start of treatment for the ovarian cancer until death from any cause. The treatment may be assessed by objective or subjective parameters; including the results of a physical examination, neurological examination, or psychiatric evaluations.

The term “progression,” as used in the context of progression of an ovarian cancer, includes the change of the cancer from a less severe to a more severe state. This could include an increase in the number or severity of tumors, the degree of metastasis, the speed with which the cancer is growing or spreading, and the like. For example, “the progression of ovarian cancer” includes the progression of such a cancer from a less severe to a more severe state, such as the progression from stage I to stage II, from stage II to stage III, etc.

“Progression free survival (PFS),” as used in the context of ovarian cancer, refers to the length of time during and after treatment of the ovarian cancer until objective tumor progression or death. The treatment may be assessed by objective or subjective parameters; including the results of a physical examination, neurological examination, or psychiatric evaluation.

“First diagnosed” refers to the initial detection of the presence of ovarian cancer in a patient, and may involve physical examination, imaging tests such as a computed tomography scan, magnetic resonance imaging scan, ultrasound, barium enema x-ray, positron emission tomography scan, or other tests such as a laparoscopy, colonoscopy, biopsy or blood test.

“Relapsed,” used synonymously with “recurrence,” refers to the return of the ovarian cancer or the signs and symptoms of ovarian cancer after a period of improvement. Recurrence of the ovarian cancer may be local or distant (metastatic).

A “clinical outcome” refers to an assessment using any endpoint indicating the status of the patient. A “positive clinical outcome” refers to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. Examples include, but are not limited to, increased overall survival time, increased occurrence of progression-free survival, reduction of tumor size or of the number of tumor cells, inhibition of tumor cell infiltration into adjacent tissues, inhibition of metastasis, decreased blood transfusion requirements, or decreased length of hospital stay. In preferred embodiments, a positive clinical outcome is increased overall survival time and/or progression-free survival. A “negative clinical outcome” refers to any failure or indicia of failure in the attenuation or amelioration of any injury, pathology or condition, including any objective or subjective parameter, as listed above.

As used herein, “discontinue” as used in the context of ovarian cancer therapy refers to ceasing or breaking the continuity of any therapy being administered.

As used herein, “monitor the progression” as used in the context of ovarian cancer therapy refers to evaluating the progression of the ovarian cancer using objective or subjective parameters including physical examination, neurological examination, psychiatric evaluation or any other accepted clinical tests.

The terms “treating” or “treatment,” used synonymously with “therapy,” refer to an approach for obtaining beneficial or desired results including but not limited to therapeutic benefit and/or a prophylactic benefit. By therapeutic benefit it is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes inhibition of tumor growth, maintenance of inhibited tumor growth, and induction of remission. Treatment methods for ovarian cancer may include surgery, chemotherapy, hormone therapy, targeted therapy or radiation therapy. In preferred embodiments, the initial treatment comprises surgery and/or chemotherapy.

“Chemotherapy” refers to the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

“Surgery” refers to surgical methods employed to remove cancerous tissue, including but not limited to tumor biopsy or removal of part or all of the colon (colostomy), bladder (cystectomy), spleen (splenectomy), gallbladder (cholecystectomy), stomach (gastrectomy), liver (partial hepatectomy), pancreas (pacreatectomy), ovaries and fallopian tubes (bilateral salpingo-oophoroectomy), omentum (omentectomy) and/or uterus (hysterectomy).

The embodiments described herein are not limited to particular methods, reagents, compounds, compositions or biological systems, which can, of course, vary.

### Methods for Detecting Proteins in a Biological Sample

Provided herein are methods for detecting proteins in a biological sample obtained from a patient with ovarian cancer. One aspect of the described methods comprises determining a level of at least three proteins in a biological sample obtained from the patient, wherein the at least three proteins are selected from ANG-2, HE4, PROSTASIN, EGFR and IL8.

The amino acid sequences of the proteins disclosed herein are well known in the art and available in public databases such as Chemical Abstracts Services Databases (e.g., the CAS Registry), GenBank, and subscription provided databases such as GenSeq (e.g., Derwent). ANG-2 (UniProtKB Swiss-Prot Accession Number 015123) is synonymous with AGPT2, ANG2, Angiopoietin 2, Angiopoetin 2A, Angiopoetin 2B, Tie2-Ligand and the like. HE4 (UniProtKB Swiss-Prot Accession Number Q14508) is synonymous with Human Epididymis Protein 4, EDDM4, Epididymal Protein 4, Epididymal Secretory Protein E4, Epididymis-Specific Whey-Acidic Protein Type Four-Disulfide Core, Major Epididymis-Specific Protein E4, Putative Protease Inhibitor WAP5, WAP Domain Containing Protein HE4-V4, WAP Four-Disulfide Core Domain 2, WAP5, and the like. PROSTASIN (UniProtKB Swiss-Prot Accession Number Q16651) is synonymous with CAP1, Channel-Activating Protease-1, PRSS8, and the like. Epidermal Growth Factor Receptor (EGFR) (UnitProtKB Swiss Prot Accession Number P00533) is synonymous with Cell Growth Inhibiting Protein-40, Cell Proliferation-Inducing Protein-61, ERBB, ERBB1, Erythroblastic Leukemia Viral V-Erb-B Oncogene Homolog, C-ErbB-1, HER1, mENA, PIG61, and the like. Interleukin 8 (IL8) (UniProtKB Swiss-Prot Accession Number P10145) is synonymous with Alveolar Macrophage Chemotactic Factor, Beta Endothelial Cell-Derived Neutrophil Activating Peptide, Beta-Thromboglobulin-Like Protein, Chemokine Ligand 8, Emoctakin, GCP1, Granulocyte Chemotactic Protein 1, LECT, LUCT, Lung Giant Cell Carcinoma-Derived Chemotactic Protein, Lymphocyte Derived Neutrophil Activating Peptide, LYNAP, Monocyte-Derived Neutrophil Chemotactic Factor, MDNCF, MONAP, Neutrophil-Activating Peptide 1, NAF, NAP1,Protein 3-10C, Small Inducible Cytokine Subfamily B Member 8, Tumor Necrosis Factor-Induced Gene 1, and the like.

ANG-2, HE4, PROSTASIN, EGFR and IL8 refer to amino acid polymers, and may include polymers in which one or more amino acid residue is an artificial chemical mimetic of a corresponding naturally occurring amino acid, as well as to naturally occurring amino acid polymers and non-naturally occurring amino acid polymers. Polypeptides of the invention may also include conservatively modified variants including polymorphic variants, interspecies homologs, and alleles. ANG-2, HE4, PROSTASIN, EGFR and IL8 further encompass all naturally occurring post-transcriptional and post-translational modifications to the polymer of amino acid residues. The claimed proteins additionally encompass all chemically, enzymatically and/or metabolically modified forms of unmodified proteins. The claimed proteins may be located in the cytoplasm of the cell, or into the extracellular milieu such as the growth medium of a cell culture. The protein may be soluble or insoluble. In preferred embodiments, the claimed polypeptides are soluble.

In some embodiments of the described methods, the ovarian cancer is a non-mucinous epithelial ovarian cancer. In some embodiments, as previously described, the biological sample is a collection of similar fluids, cells, or tissues (e.g., surgically resected tumor tissue, biopsies, including fine needle aspiration), isolated from a subject, as well as fluids, cells, or tissues present within a subject. The biological sample assessed for the presence of the selected proteins may be urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like. In preferred embodiments, the biological sample is serum, plasma or ascites.

Suitable assays for the detection of levels of biomarkers include, but should not be limited to, western blot analysis, radioimmunoassay, immunofluorimetry, immunoprecipitation, immunodiffusion, electrochemiluminescence (ECL) immunoassay, immunohistochemistry, fluorescence-activated cell sorting (FACS) or ELISA assay. In preferred embodiments, the level of the at least three proteins is determined using an electrochemiluminescence (ECL) immunoassay.

In some embodiments, the levels of EGFR, HE4 and IL8 are determined. In other embodiments, the levels of ANG-2, HE4, PROSTASIN, EGFR and IL8 are determined.

### Methods for Calculating a Quantitative Score for a Patient With Ovarian Cancer

Provided herein are methods for calculating a quantitative score for a patient with ovarian cancer. These methods comprise determining a level of at least three proteins in a biological sample obtained from the patient, wherein the at least three proteins are selected from ANG-2, HE4, PROSTASIN, EGFR and IL8, and calculating a quantitative score for the patient by weighting the level of the at least three proteins by their contribution to a clinical outcome.

In some embodiments, the ovarian cancer is a non-mucinous epithelial ovarian cancer. In some embodiments, the biological sample is serum, plasma, or ascites. In some embodiments the level of at least three proteins is determined using an immunoassay. In some embodiments, the immunoassay is an electrochemiluminescent assay.

In some embodiments, the levels of ANG-2, HE4, PROSTASIN, EGFR and IL8 are determined. As used herein, a “quantitative score” is the mathematically calculated numerical value representing the hazard at time (t), or the instantaneous rate of occurrence of an event. In some embodiments, a quantitative score may be calculated using an algorithm with overall survival as the outcome, and in some embodiments a quantitative score may be calculated using an algorithm with progression free survival as the outcome. The algorithms may be generated using methods known in the art and as described in standard textbooks on survival analysis (David G. Kleinbaum and Mitchel Klein (2011). Survival Analysis: A Self-Learning Text, Third Edition. Springer). A quantitative score may be calculated by first log-transforming the levels of a selection of biomarkers to mitigate the effect of outliers. Survival analysis methods that may be employed to generate a quantitative score include Kaplan-Meier plots, log-rank tests, Cox proportional hazards regression analysis, and tests of residuals and proportional hazards assumptions. Values may be reported both unadjusted and adjusted for multiple comparisons using the Benjamini-Hochberg procedure. In some embodiments, the analysis may take into account clinical variables including STLENRM (length of first remission), STROUTE (route of administration of therapy), STPLNTX (planned therapy on study), STREGN (geographical region where the patient was from/treated).

In some embodiments, the levels of ANG-2, HE4, PROSTASIN, EGFR and IL8 are determined. In some embodiments, the quantitative score is calculated based on the algorithm:

h_{OS}(t)=h0_{OS}(t) exp(˜A*ANG2+˜B*HE4+˜C*PROSTASIN−˜D*EGFR+˜E*IL8)

wherein h_{OS}(t) is the hazard at time (t) and h0_{OS}(t) is the baseline hazard with overall survival as the outcome, wherein the gene symbols in the equation represent the protein levels, and wherein coefficients A, B, C, D, and E are the coefficients derived for each respective protein, the model being optimized to provide maximal prognostic information for the given population of ovarian cancer patients. In some embodiments, the quantitative score is calculated based on the algorithm:

h_{OS}(t)=h0_{OS}(t) exp(1.213 ANG2+0.171 HE4+0.102 PROSTASIN−1.406 EGFR+0.207 IL8).

It is understood to those of skill in the art that the coefficients of the quantitative score equations provided herein are subject to some variability depending upon the population of subjects diagnosed with ovarian cancer.

In other embodiments, the quantitative score is calculated based on the algorithm:

h_{PFS}(t)=h0_{PFS}(t) exp(˜A*ANG2+˜B*HE4+˜C*PROSTASIN−˜D*EGFR+˜E*IL8)

wherein h_{PFS}(t) is the hazard at time (t) and h0_{PFS}(t) is the baseline hazard with progression free survival as the outcome, wherein the gene symbols in the equation represent the protein levels, and wherein coefficients A, B, C, D, and E are the coefficients derived for each respective protein, the model being optimized to provide maximal prognostic information for the given population of ovarian cancer patients. In some embodiments, the quantitative score is calculated based on the algorithm:

h_{PFS}(t)=h0_{PFS}(t) exp(0.077 ANG2+0.123 HE4+0.008 PROSTASIN−0.545 EGFR+0.156 IL8).

In some embodiments, the levels of EGFR, HE4 and IL8 are determined. In some embodiments, the quantitative score is calculated based on the algorithm:

h_{OS}(t)=h0_{OS}(t) exp(˜A*HE4−˜B*EGFR+˜C*IL8)

wherein h_{OS}(t) is the hazard at time (t) and h0_{OS}(t) is the baseline hazard with overall survival as the outcome, wherein the gene symbols in the equation represent the protein levels, and wherein coefficients A, B, and C are the coefficients derived for each respective protein, the model being optimized to provide maximal prognostic information for the given population of ovarian cancer patients. In some embodiments, the quantitative score is calculated based on the algorithm:

h_{OS}(t)=h0_{OS}(t) exp(0.234 HE4−1.464 EGFR+0.273 IL8)

It is understood to those of skill in the art that the coefficients of the quantitative score equations provided herein are subject to some variability depending upon the population of subjects diagnosed with ovarian cancer.

In other embodiments, the quantitative score with progression-free survival as the outcome is based on the algorithm:

h_{PFS}(t)=h0_{PFS}(t) exp(˜A*HE4−˜B*EGFR+˜C*IL8)

wherein h_{PFS}(t) is the hazard at time (t) and h0_{PFS}(t) is the baseline hazard with overall survival as the outcome, wherein the gene symbols in the equation represent the protein levels, and wherein coefficients A, B, and C are the coefficients derived for each respective protein, the model being optimized to provide maximal prognostic information for the given population of ovarian cancer patients. In some embodiments, the quantitative score is calculated based on the algorithm:

h_{PFS}(t)=h0_{PFS}(t) exp(0.124 HE4−0.538 EGFR+0.161 IL8).

Because the protein levels in the described algorithms are log₂ transformed, one unit increase corresponds to doubling the value in the original scale. In the described algorithms, an increase in EGFR corresponds to a decrease in risk whereas an increase in ANG2, HE4, PROSTASIN or IL8 corresponds to an increase in risk.

It is to be understood that the numerical parameters set forth in the described algorithms and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

### Methods for Predicting a Likelihood of a Clinical Outcome

Provided herein are methods for predicting a likelihood of a clinical outcome in a patient with ovarian cancer. These methods comprise determining a level of at least three proteins in a biological sample obtained from the patient, wherein the at least three proteins are selected from ANG-2, HE4, PROSTASIN, EGFR and IL8, calculating a quantitative score for the patient by weighting the level of the at least three proteins by their contribution to a clinical outcome, and predicting a likelihood of a clinical outcome for the patient based on the quantitative score.

In some embodiments, an increase in the quantitative score correlates with a decreased likelihood of a positive clinical outcome, wherein a decrease in the quantitative score correlates with an increased likelihood of a positive clinical outcome. As previously described, a “positive clinical outcome” refers to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. Examples include, but are not limited to, increased overall survival time, increased occurrence of progression-free survival, reduction of tumor size or of the number of tumor cells, inhibition of tumor cell infiltration into adjacent tissues, inhibition of metastasis, decreased blood transfusion requirements, or decreased length of hospital stay. In preferred embodiments, a positive clinical outcome is increased overall survival time and/or progression-free survival. A “negative clinical outcome” refers to any failure or indicia of failure in the attenuation or amelioration of any injury, pathology or condition, including any objective or subjective parameter, as listed above.

In some embodiments, a likelihood of a negative clinical outcome for the patient informs a decision to discontinue current ovarian cancer therapy and/or initiate an ovarian cancer therapy, and wherein a likelihood of a positive clinical outcome for the patient informs a decision to monitor the progression of the ovarian cancer and/or continue current ovarian cancer therapy. As previously described, “discontinue” refers to ceasing or breaking the continuity of any therapy being administered, and “monitor the progression” refers to evaluating the progression of the ovarian cancer using objective or subjective parameters including physical examination, neurological examination, psychiatric evaluation or any other accepted clinical tests. In some embodiments, the positive clinical outcome is increased overall survival time. In some embodiments, the positive clinical outcome is progression free survival.

In some embodiments, the ovarian cancer is a non-mucinous epithelial ovarian cancer. In some embodiments, the likelihood of a clinical outcome is predicted when the ovarian cancer is first diagnosed. In other embodiments, the likelihood of a clinical outcome is predicted when the ovarian cancer relapses for the first time 6 to 24 months after an initial treatment. In further embodiments, the likelihood of a clinical outcome is predicted when the ovarian cancer relapses at any time after an initial treatment. In still further embodiments, the likelihood of a clinical outcome is predicted at any time after a first diagnosis. In some embodiments, the initial treatment comprises surgery and/or chemotherapy. As previously described, “chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository. Also as previously described, “surgery” refers to surgical methods employed to remove cancerous tissue, including but not limited to tumor biopsy or removal of part or all of the colon (colostomy), bladder (cystectomy), spleen (splenectomy), gallbladder (cholecystectomy), stomach (gastrectomy), liver (partial hepatectomy), pancreas (pacreatectomy), ovaries and fallopian tubes (bilateral salpingo-oophoroectomy), omentum (omentectomy) and/or uterus (hysterectomy).

In some embodiments, the biological sample is serum, plasma, or ascites. Also disclosed are methods of predicting a likelihood of a clinical outcome in a patient with ovarian cancer, wherein the level of at least three proteins is determined using an immunoassay.

In some embodiments, the levels of ANG-2, HE4, PROSTASIN, EGFR and IL8 are determined and in further embodiments, the quantitative score is calculated based on the algorithms described herein. In other embodiments, the levels of EGFR, HE4 and IL8 are determined and in further embodiments, the quantitative score is calculated based on the algorithms described herein.

### Reagents and Kits of the Invention

Provided herein are sets of reagents to measure the levels of three or more biomarkers in a patient with ovarian cancer wherein the biomarkers comprise ANG2, EGFR, HE4, IL8 and PROSTASIN and their measurable fragments as well as test kits comprising the described sets of reagents. In some embodiments, the sets of reagents include binding molecules. In preferred embodiments, the binding molecules for detecting a set of biomarkers described herein are antibodies, or an antigen-binding fragment thereof The provided antibody, or antigen-binding fragment, may be in solution, lyophilized, affixed to a substrate, carrier, or plate, or conjugated to a detectable label.

The described kits may also include additional components useful for performing the methods described herein. By way of example, the kits may comprise means for obtaining a sample from a subject, a control sample, e.g., a sample from a subject having slowly progressing cancer and/or a subject not having cancer, one or more sample compartments, and/or instructional material which describes performance of a method of the invention and tissue specific controls/standards.

The means for determining the level of the described biomarkers can further include, for example, buffers or other reagents for use in an assay for determining the level of the claimed biomarkers. The instructions can be, for example, printed instructions for performing the assay and/or instructions for evaluating the level of expression of the described biomarkers.

The described kits may also include means for isolating a sample from a subject. These means can comprise one or more items of equipment or reagents that can be used to obtain a fluid or tissue from a subject. The means for obtaining a sample from a subject may also comprise means for isolating blood components, such as serum, from a blood sample. Preferably, the kit is designed for use with a human subject.

The described kits may also include a blocking reagent that can be applied to a sample to decrease nonspecific binding of a primary or secondary antibody. An example of a blocking reagent is bovine serum albumin (BSA), which may be diluted in a buffer prior to use. Other commercial blocking reagents, such as Block Ace and ELISA Synblock (AbD serotec), Background Punisher (BIOCARE MEDICAL), and StartingBlock™ (Thermo Fisher Scientific) are known in the art. The described kits may also include a negative control primary antibody that does not bind to the described biomarkers sufficiently to yield a positive result in an antibody-based detection assay. In addition, the described kits may include a secondary antibody capable of binding to a primary antibody. In some embodiments the secondary antibody may be conjugated to a detectable label, such as horse radish peroxidase (HRP) or a fluorophore, to allow for detection of the primary antibody bound to a sample. The described kits may also include a colorimetric or chemiluminescent substrate that allows the presence of a bound secondary antibody to be detected on a sample. In some embodiments the colorimetric or chemiluminescent substrate may be 2,2′-azino-bis(3-ethylbenzothiazoline-6-sulphonic acid) (ABTS); 3,3′,5,5′-Tetramethylbenzidine (TMB); 3,3′-Diaminobenzidine (DAB); SuperSignal® (Thermo Fisher Scientific); ECL reagent (Thermo Fisher Scientific) or other such reagents known to those of ordinary skill in the art.

The following examples are provided to supplement the prior disclosure and to provide a better understanding of the subject matter described herein. These examples should not be considered to limit the described subject matter. It is to be understood that the examples and embodiments described herein are for illustrative purposes only and that various modifications or changes in light thereof will be apparent to persons skilled in the art and are to be included within the and can be made without departing from the true scope of the invention.

### EXAMPLES

### Example 1

### Selection of Patient Population

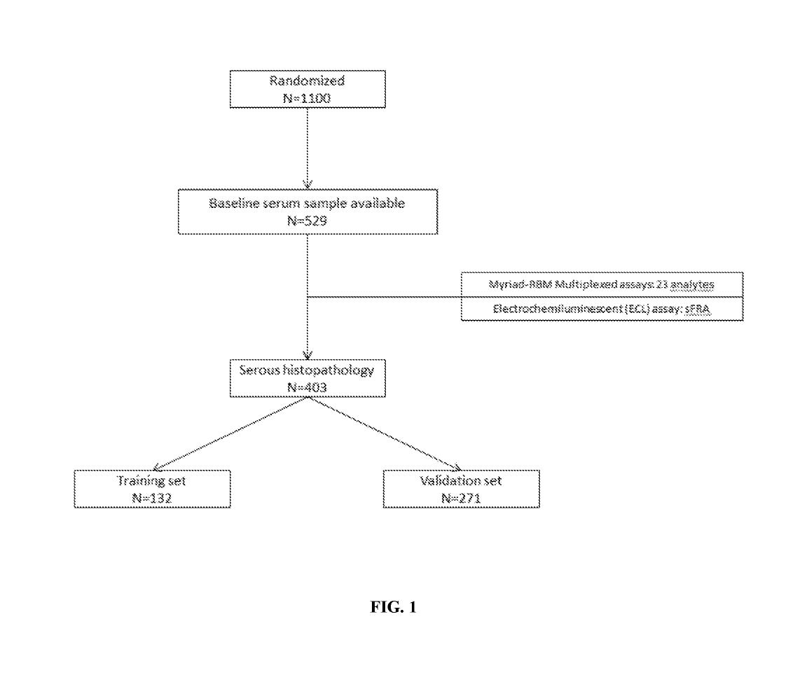

A total of 1100 women with non-mucinous EOC who had relapsed 6 to 24 months after initial surgery and platinum/taxane chemotherapy were enrolled and randomized to chemotherapy plus either farletuzumab or placebo. A total of 529 patients consented to a translational sub-study and pre-treatment baseline serum samples were available for analysis in the present study; 403 subjects were in the serous sub-group examined herein. For the analyses presented, the 403 subjects were analyzed in 2 cohorts representing 132 subjects from the placebo group (the training set) and 271 subjects from the farletuzumab-treated group (the validation set) (FIG. 1).

### Example 2

### Serum Biomarker Assays

Serum proteins were assessed on baseline (pre-treatment) serum samples for all subjects enrolled in the translational sub-study. Serum folate receptor alpha (FRA) was measured by Electrochemiluminescent (ECL) assays as previously described (O'Shannessy et al., J Ovarian Res, 6(1):29 (2013)). All other markers were measured using Luminex® multiplexed assays at Myriad-RBM. Briefly, serum samples were thawed at room temperature, vortexed, spun for clarification and loaded into a master microtiter plate. Individual sample aliquots were introduced into one of the capture microsphere multiplexes of the Multi-Analyte PROFILE-Ov (MAP) followed by thorough mixing of the sample and capture microspheres prior to incubation for 1 hour at room temperature. Multiplexed cocktails of biotinylated reporter antibodies for each multiplex were added and incubated for an additional hour at room temperature. Multiplexes were developed using an excess of streptavidin-phycoerythrin solution following an incubation period of 1 hour at room temperature, where after the volume of each multiplexed reaction was reduced by vacuum filtration prior to analysis using a Luminex® instrument. The resulting data stream was interpreted using data analysis software developed by Myriad RBM. For each multiplex, both calibrators and controls were included on each micro-titer plate. Standard curve, control and sample quality control (QC) were performed to ensure proper assay performance. Study sample values for each of the analytes localized in a specific multiplex were determined using 4 and 5 parameter, weighted and non-weighted curve fitting algorithms.

### Example 3

### Statistical Methods

Because most analytes were right-skewed and also to mitigate the effect of outliers, log₂ transformed variables were used throughout this analysis (Table 1). All analyses were performed using R version 3.0 or higher. Two-sided p-values <0.05 were considered significant. P-values are reported both unadjusted and adjusted for multiple hypothesis testing.

TABLE 1

| | | | Log₂-transformed Data | |
|---|---|---|---|---|
| | Untransformed Data | | | Standard |
| Analyte | Mean | Standard Deviation | Mean | Deviation |
| Prostasin | 403.16 | 343.47 | 8.36 | 0.85 |
| EGFR | 4.09 | 0.72 | 2.05 | 0.26 |
| IL_10 | 6.04 | 6.99 | 2.14 | 1.12 |
| sFRA | 1697.73 | 3215.17 | 10.02 | 1.21 |
| IGFBP_2 | 185.17 | 102.32 | 7.32 | 0.82 |
| IL_8 | 21.94 | 38.42 | 3.74 | 1.25 |
| VEGF_D | 332.19 | 227.58 | 8.16 | 0.7 |
| IL_18 | 236.63 | 146.99 | 7.67 | 0.78 |
| CA_72_4 | 156.89 | 247.12 | 6.05 | 1.95 |
| IL_6 | 9.5 | 29.53 | 1.77 | 1.47 |
| MSLN | 118.66 | 86.78 | 6.51 | 1.08 |
| TN_C | 763.69 | 646.85 | 9.26 | 0.96 |
| VEGFR_1 | 487.99 | 1064.43 | 8.31 | 0.89 |
| AR | 194.24 | 98.85 | 7.39 | 0.83 |
| PDGF_BB | 12485.67 | 6383.42 | 13.31 | 1.23 |
| hCG | 2.78 | 2.47 | 1.1 | 1.12 |
| ANG_2 | 5.23 | 2.67 | 2.28 | 0.59 |
| HE4 | 504.76 | 922.07 | 7.61 | 1.88 |
| CA125 | 591.71 | 1427.17 | 7.55 | 2.18 |
| MCP_1 | 413.63 | 220.24 | 8.48 | 0.83 |
| MIF | 0.47 | 1.35 | −1.58 | 1.11 |
| Kallikrein_5 | 16.16 | 56.06 | 2.51 | 1.55 |
| CA_19_9 | 17.25 | 32.49 | 3.08 | 1.71 |
| NSE | 4.69 | 12.32 | 1.31 | 1.25 |

Survival analysis methods used in these analyses, including Kaplan-Meier plots, log-rank tests, Cox proportional hazards regression analysis, and tests of residuals and proportional hazards assumptions are described in standard textbooks on survival analysis (David G. Kleinbaum and Mitchel Klein (2011). Survival Analysis: A Self-Learning Text, Third Edition. Springer). Values are reported both unadjusted and adjusted for multiple comparisons using the Benjamini-Hochberg procedure. Survival analyses were performed using the R package “survival”. Lasso Cox PH analyses were performed using the R package “glmnet”. C-statistics (AUC) were calculated using the method of Heagerty, Lumley and Pepe 2000 (Time-Dependent ROC Curves for Censored Survival Data and a Diagnostic Marker: Patrick J. Heagerty, Thomas Lumley and Margaret S. Pepe. Biometrics, 2000, vol. 56, issue 2, pages 337-344) using the R package “survivalROC”. Bootstrap and cross-validation analyses were implemented in base R.

The outcome variables assessed were progression free survival (PFS) and overall survival (OS). For each patient, the observed survival time is either time to failure, T, or censoring time, C, whichever comes first. A binary event indicator, δ, indicates whether the observed time is time to failure (δ=1, indicating the event has occurred) or censoring time (δ=0, indicating that the event occurs after the follow up period). The survival analysis models were aimed at making inference regarding the time from the origin to the event of interest. To this end, the Kaplan-Meier (KM) estimator, which is a nonparametric approach, and Cox Proportional Hazard (CPH) regression model, which is semi-parametric, methods were used.

### Example 4

### Univariate Analyses

A total of 24 serum protein analytes previously described in the literature as prognostic for EOC were initially assessed for prognosis in the 132-subject training set in the present study. Univariate analyses for OS and PFS confirmed a number of these markers as significant individual prognostic markers (Table 2). Table 2 shows the p-values for all analytes from univariate Cox proportional hazards models for PFS and OS in the training set (n=132). P-values are also adjusted for multiple testing. In Table 2, p-value represents the unadjusted p-value and q-value represents p-values adjusted for multiple testing.

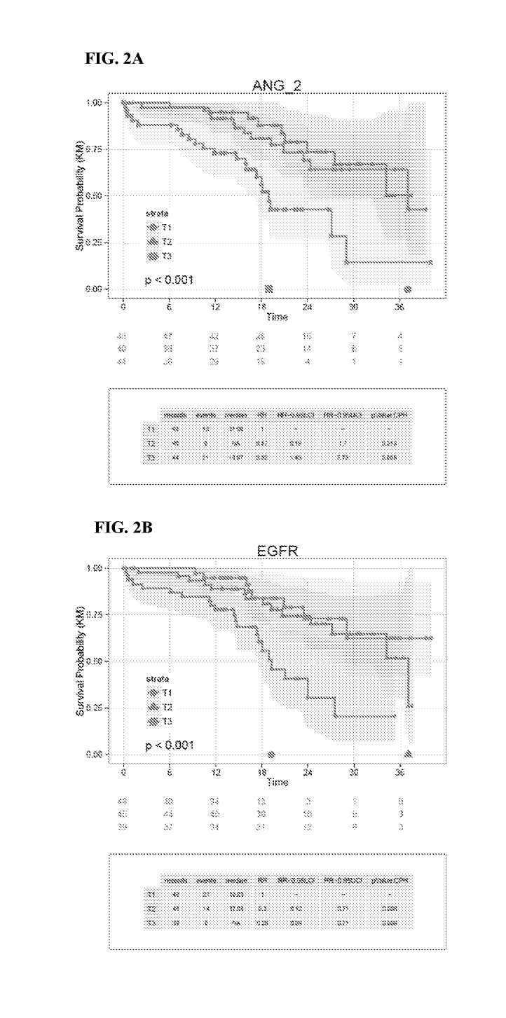

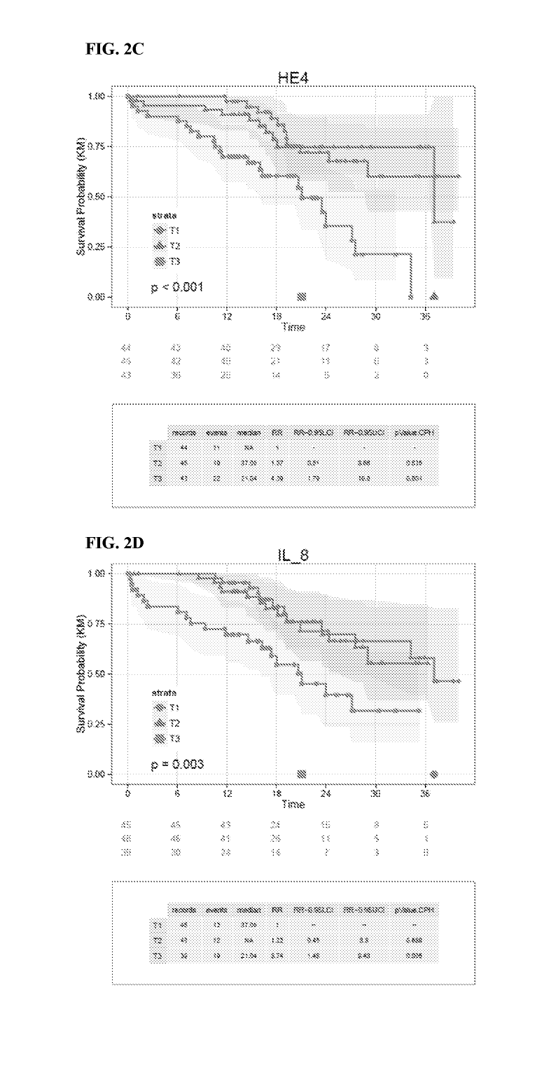

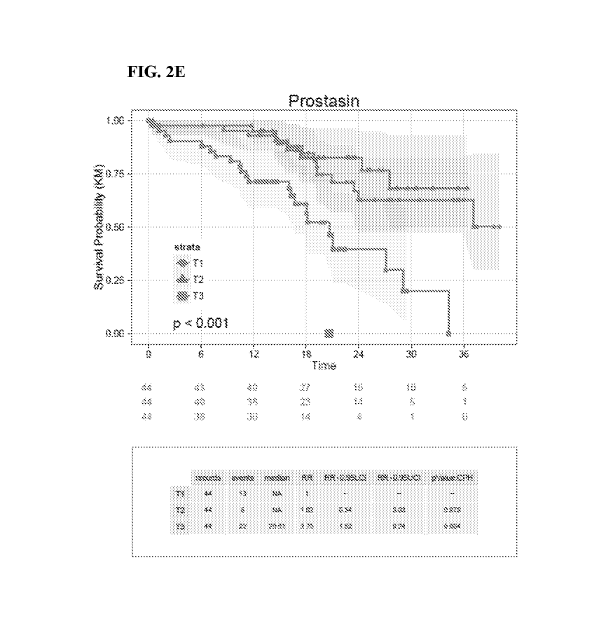

The Kaplan-Meier (KM) plots for the top 5 performing individual markers (all with p<0.005) for prognosis for OS are shown in FIG. 2A (ANG-2), FIG. 2B (EGFR), FIG. 2C (HE4), FIG. 2D (IL8) and FIG. 2E (PROSTASIN). The 4 most significant analytes, ANG-2, EGFR, HE4 and Prostasin, all remained significant when adjusted for multiple comparisons (denoted as q-values; q<0.0005). The prognostic effect is clearly evident in the KM plots (log-rank p<0.001) for each of the 4 most significant markers based on tertiles. For HE4, PROSTASIN, IL-8 and ANG-2, higher values are prognostic of higher mortality risk whereas for EGFR higher values are prognostic of lower risk. Further, the KM plots suggest a threshold for the prognostic effect for each protein: the 1^{st} tertile for EGFR, and the 3^{rd} tertile for ANG-2, HE4 and PROSTASIN.

HE4, EGFR and PROSTASIN were also identified as top performing markers for PFS (Table 2), although not statistically significant when corrected for multiple comparisons. Similar effects to those seen with OS are seen using PFS as the outcome where higher levels of HE4 and PROSTASIN indicate poor prognosis whereas higher levels of EGFR indicate better prognosis. The more striking results obtained using OS as the outcome, relative to PFS, may reflect, in part, the unambiguous nature of an OS outcome in contrast to a clinically defined progression event for which a certain degree of subjectivity is involved.

TABLE 2

| | |
|---|---|
| OS | PFS |

| Analyte | p-value | q-value | p-value | q-value |
|---|---|---|---|---|
| ANG_2 | <0.00001 | 0.00007 | 0.22978 | 0.55147 |
| AR | 0.35811 | 0.45235 | 0.92818 | 0.94543 |
| CA_19_9 | 0.21804 | 0.29072 | 0.82151 | 0.94338 |
| CA_72_4 | 0.07998 | 0.13947 | 0.69516 | 0.94338 |
| CA125 | 0.07453 | 0.13947 | 0.02751 | 0.16503 |
| EGFR | 0.00005 | 0.00044 | 0.0207 | 0.16503 |
| hCG | 0.83385 | 0.90932 | 0.31662 | 0.58427 |
| HE4 | 0.00006 | 0.00044 | 0.00305 | 0.07322 |
| IGFBP_2 | 0.00323 | 0.00988 | 0.37082 | 0.59331 |
| IL_10 | 0.00169 | 0.00674 | 0.09158 | 0.31399 |
| IL_18 | 0.01002 | 0.02673 | 0.05456 | 0.21823 |
| IL_6 | 0.07209 | 0.13947 | 0.34083 | 0.58427 |
| IL_8 | 0.00113 | 0.00544 | 0.01098 | 0.13172 |
| Kallikrein_5 | 0.96972 | 0.96972 | 0.8194 | 0.94338 |
| MCP_1 | 0.87143 | 0.90932 | 0.80528 | 0.94338 |
| MIF | 0.39557 | 0.47468 | 0.10962 | 0.32885 |
| MSLN | 0.08136 | 0.13947 | 0.26601 | 0.58037 |
| NSE | 0.15697 | 0.23546 | 0.2296 | 0.55147 |
| PDGF_BB | 0.73508 | 0.84009 | 0.33643 | 0.58427 |
| Prostasin | 0.00007 | 0.00044 | 0.03551 | 0.17047 |
| sFRA | 0.0508 | 0.12193 | 0.82546 | 0.94338 |
| TN_C | 0.21565 | 0.29072 | 0.89348 | 0.94543 |
| VEGF_D | 0.00329 | 0.00988 | 0.46981 | 0.70472 |
| VEGFR_1 | 0.12349 | 0.19759 | 0.94543 | 0.94543 |

### Example 5

### Multivariate Analyses

### Lasso Variable Selection

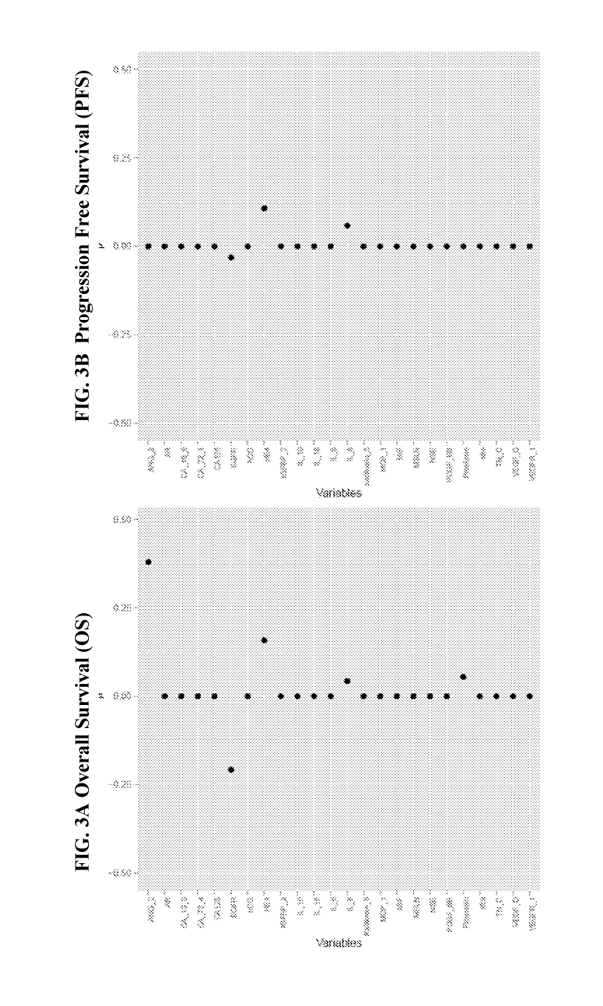

A lasso variable selection procedure considering all 24 serum protein analytes as candidates was used to construct multivariable CPH models using the 132 subjects in the training set. The lasso procedure shrinks the coefficients towards zero and imposes sparsity by forcing some of the coefficients to become exactly zero (i.e., excluded from the model). To determine the appropriate level of sparsity, 10-fold cross validation was used. Depending on whether OS (FIG. 3A) or PFS (FIG. 3B) is used as the outcome, lasso identified either 5 or 3 analytes respectively. Both models, i.e., PFS-derived and OS-derived, were fitted to the observed data with OS or PFS as the outcome.

### Model MI

Using PFS as the outcome, the lasso procedure selected 3 analytes, namely, HE4, EGFR and IL-8. Using a CPH model with these three analytes results in two different estimations of hazard depending on whether OS or PFS is used as the outcome:

h_{OS}(t)=h0_{OS}(t)exp(0.234 HE4−1.464 EGFR+0.273 IL8)

h_{PFS}(t)=h0_{PFS}(t)exp(0.124 HE4−0.538 EGFR+0.161 IL8)

The models present the hazard at time h(t) based on the baseline hazard h0(t). Note that analytes in these models are log₂ transformed. Therefore, one unit increase corresponds to doubling the value in the original scale. For example, doubling HE4 results in 13% (exp(0.124)=1.13) increase in risk (keeping all other analytes constant) with respect to progression free survival.

### Model M2

Using OS as the outcome, the lasso procedure selected 5 analytes, namely, HE4, EGFR, PROSTASIN, IL-8 and ANG-2. Including these variables in a CPH model the hazard function for OS and PFS is estimated separately as:

h_{OS}(t)=h0_{OS}(t) exp(1.213 ANG2+0.171 HE4+0.102 PROSTASIN−1.406 EGFR+0.207 IL8)

h_{PFS}(t)=h0_{PFS}(t) exp(0.077 ANG2+0.123 HE4−0.008 PROSTASIN−0.545 EGFR+0.156 IL8)

In this case, doubling the value of HE4 is associated with increasing the risk with respect to overall survival by 19% (exp(0.171)=1.19), keeping all other analytes constant. That is, the relative risk is 1.19 when HE4 doubles and everything else remains the same.

Note that in both models, i.e. M1 and M2, an increase in EGFR corresponds to a decrease in risk whereas an increase in any of the other analytes corresponds to an increase in risk.

For both the M1 and M2 models, tests for violation of the proportional hazards assumption for each analyte were not significant, martingale residuals indicated that the linear functional form was appropriate, and deviance residuals indicated that there were no significant outliers, indicating the fitness of the analyses and models.

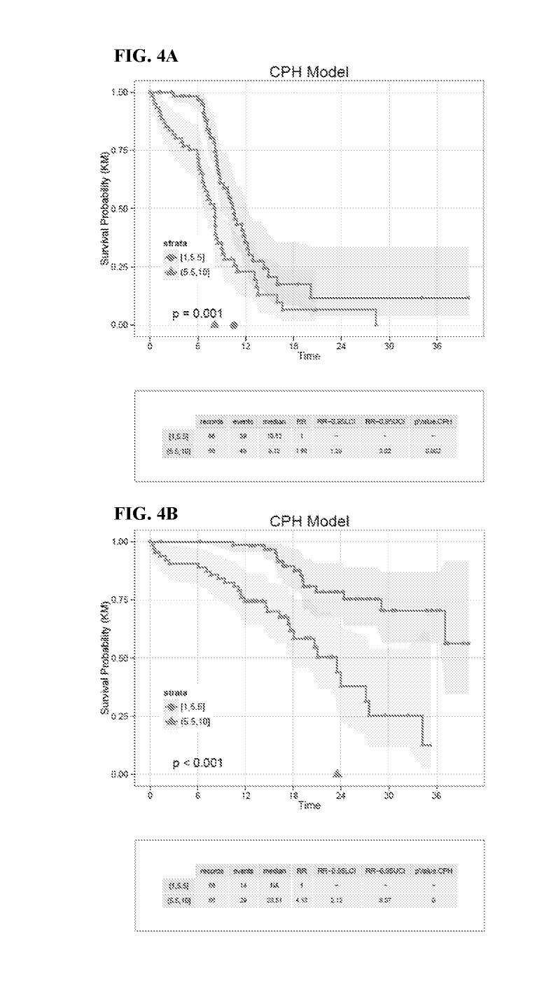

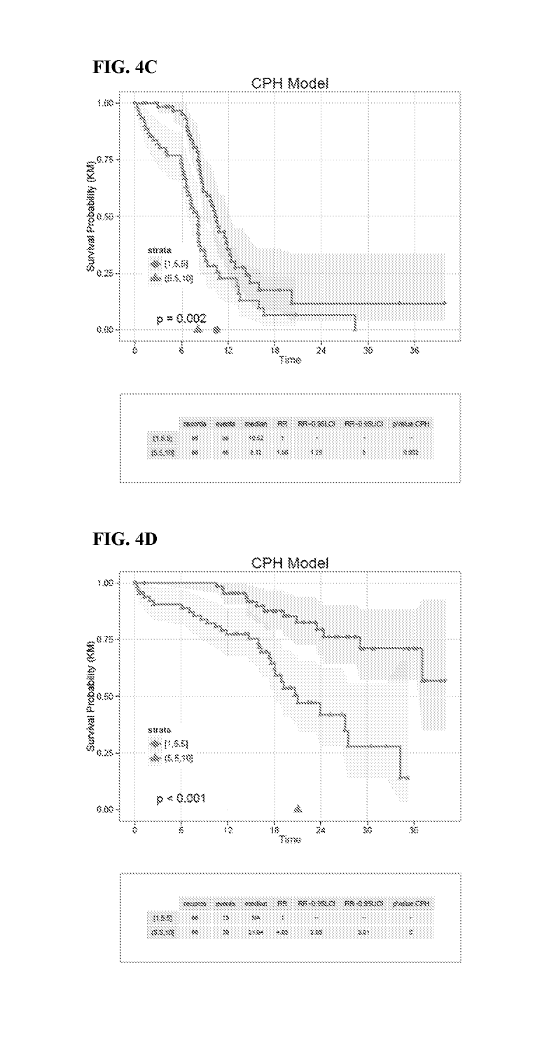

The linear combination of analytes (shown inside the exponent part of the models) was used to create a risk score for each subject. The score is normalized to be between 1 and 10, where a score of 10 represents the highest level of risk. Using this approach, KM estimates of survival functions for OS and PFS (PROFILE-Ov), as median splits, are presented in FIG. 4A, FIG. 4B, FIG. 4C, and FIG. 4D. FIG. 4A shows the KM plot for the PFS-derived model with PS as the outcome, FIG. 4B shows the KM plot for the PFS-derived model with OS as the outcome, FIG. 4C shows the KM plot for the PFS-derived model with PFS as the outcome and FIG. 4D shows the KM plot for the PFS-derived model with OS as the outcome. The M1 model performs better (provides a better separation of high vs. low risk) than the M2 model for both PFS and OS with HR=1.98 (log-rank p<0.001) and HR=4.13 (log-rank p<0.001), respectively. Further, a 10-fold cross validation was performed to compare the two models (M1 and M2) in terms of the area under the ROC curve by setting cutoffs of 12 months for PFS and 24 months for OS. For the M1 model the average AUC for OS and PFS were 0.762±0.062 and 0.610±0.085. For the M2 model the corresponding averages were 0.748±0.093 and 0.595±0.081.

Note that the 3 analytes identified by modeling PFS as the outcome (M1)—specifically, HE4, EGFR and IL-8—were contained within the 5 analytes identified using OS as the outcome measure (M2). Importantly, neither model is dependent on the order in which the analytes are measured. Overall the M1 model, hereinafter termed PROFILE-Ov, outperforms the M2 model, using fewer variables, and was therefore progressed for further analysis, including that of the validation cohort.

The PROFILE-Ov score remained statistically significant even after controlling for several clinical variables known to be relevant to prognosis (Table 3) including BRCA status, Karnofsky Performance Status (KPS) and length of first remission. Controlling for clinical variables, the hazard ratio with respect to OS between two subjects with one unit difference in PROFILE-Ov score is 1.377 (p=8.46×10⁻⁶). That is, one unit increase in score coincides with 38% increase in risk. For example, HR is 38% higher for a subject with score 6 compared to a subject with score 5. The corresponding hazard ratio with respect to PFS is 1.155 (p=0.001).

Since the samples used in the present study were derived from a farletuzumab clinical trial, the PROFILE-Ov score derived using only the placebo group (training cohort) was further assessed for potential interaction with treatment. Using the placebo group as the baseline, the p-values for the interaction terms with low dose and high dose farletuzumab arms were 0.405 and 0.645 respectively. Since no interaction was evident for either farletuzumab arm, both arms (low dose and high dose) were combined and used as a validation cohort for evaluation of the PROFILE-Ov model.

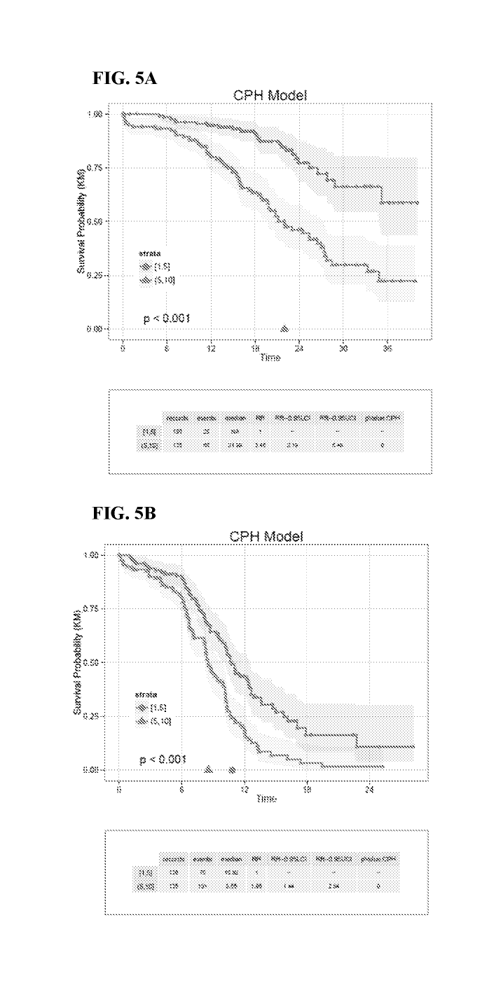

FIG. 5A and FIG. 5B present KM analyses for the validation cohort for both PFS and OS by median split for PROFILE-Ov. FIG. 5A shows the KM plot for the PFS-derived model with PFS as the outcome and FIG. 5B shows the KM plot for the PFS-derived model with OS as the outcome. Both PFS and OS differ significantly (log-rank p<0.001) with HRs of 1.95 and 3.46, respectively. Note that the 95% confidence intervals for OS are essentially non-overlapping, a reflection of the power of the separation achieved with this model. Importantly, the PROFILE-Ov score remains significant in a multivariate CPH model controlling for clinical variables with HR=1.094 and p=0.015 (Table 3). Note that the results for the validation set are based on the model developed and fitted to the training set. That is, the model was not re-optimized based on the validation set. As can be seen, PROFILE-Ov performs very well on a dataset it has not previously seen.

TABLE 3

| | PFS | | | | | OS | | | | |
|---|---|---|---|---|---|---|---|---|---|---|
| | | exp | se | | | | exp | se | | |
| Variable | coef | (coef) | (coef) | z | Pr(>\|z\|) | coef | (coef) | (coef) | z | Pr(>\|z\|) |
| Age | −0.010 | 0.990 | 0.008 | −1.250 | 0.211 | −0.003 | 0.997 | 0.011 | −0.257 | 0.797 |
| Race (Caucasian) | 0.322 | 1.380 | 0.225 | 1.432 | 0.152 | 0.410 | 1.506 | 0.314 | 1.306 | 0.192 |
| Length of | −0.338 | 1.402 | 0.184 | 1.836 | 0.066 | 0.571 | 1.770 | 0.273 | 2.095 | 0.036 |
| Remission 6-12 | | | | | | | | | | |
| Length of | −0.144 | 0.866 | 0.253 | −0.570 | 0.569 | 0.740 | 2.096 | 0.359 | 2.060 | 0.039 |
| Remission 18-24 | | | | | | | | | | |
| Planned Tx (Paclataxel) | 0.186 | 1.204 | 0.220 | 0.842 | 0.400 | 0.270 | 1.309 | 0.309 | 0.873 | 0.382 |
| Geography | 0.161 | 1.174 | 0.207 | 0.776 | 0.438 | 0.562 | 1.755 | 0.280 | 2.009 | 0.045 |
| Route of admin (IV) | 0.166 | 1.181 | 0.275 | 0.604 | 0.546 | 0.387 | 1.472 | 0.408 | 0.948 | 0.343 |
| KPS | −0-030 | 0.970 | 0.012 | −2.433 | 0.015 | −0.046 | 0.955 | 0.015 | −3.126 | 0.002 |
| Albumin < ULN | −0.353 | 0.703 | 0.280 | −1.259 | 0.208 | −0.622 | 0.537 | 0.316 | −1.966 | 0.049 |
| Liver lesions (Yes) | 0.626 | 1.871 | 0.167 | 3.743 | 0.0002 | 0.659 | 1.933 | 0.227 | 2.901 | 0.004 |
| Ascites (Yes) | −0.214 | 0.807 | 0.227 | −0.943 | 0.346 | 0.121 | 1.129 | 0.289 | 0.419 | 0.675 |
| PROFILE-Ov | 0.086 | 1.090 | 0.033 | 2.640 | 0.008 | 0.153 | 1.165 | 0.048 | 3.209 | 0.001 |

| | | |
|---|---|---|
| Model | Concordance = 0.651 (se = 0.025) | Concordance = 0.723 (se = 0.033) |
| Statistics | Likelihood ratio test = 46.31 on 12 | Likelihood ratio test = 57.28 on 12 |
| | df, p = 6.141e−06 | df, p = 7.024e−08 |
| | Wald test = 44.39 on 12 df, | Wald test = 52.75 on 12 df, |
| | p = 1.309e−05 | p = 4.568e−07 |
| | Score (logrank) test = 46.37 on | Score (logrank) test = 59.24 on |
| | 12 df, p = 5.99e−06 | 12 df, p = 3.11e−08 |

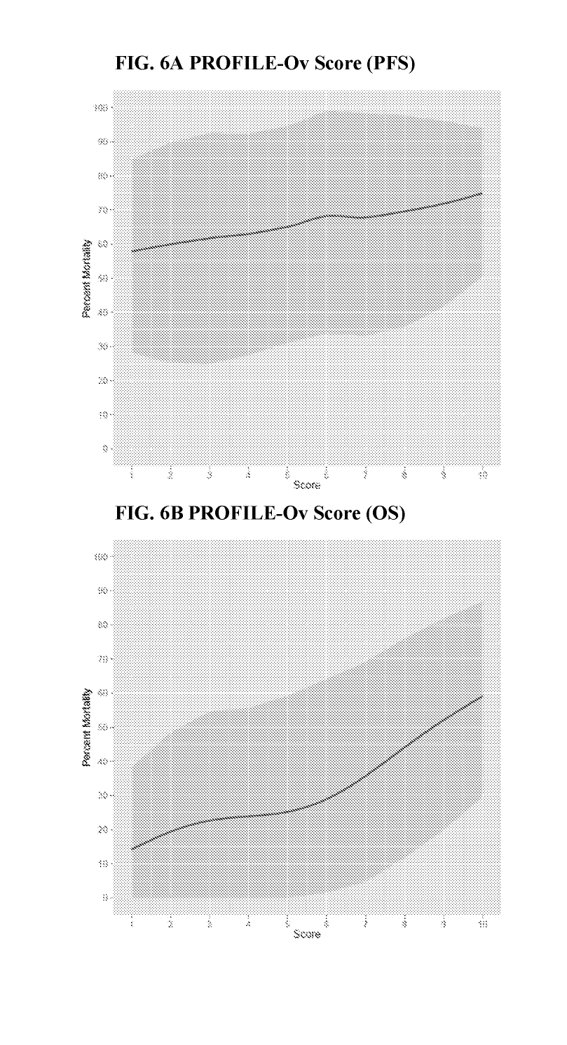

FIG. 6A and FIG. 6B show plots of the PROFILE-Ov scores from the PFS model (FIG. 6A) and from the OS model (FIG. 6B) divided into 10 equal intervals and plotted against the observed, not estimated, mortality rate. The observed mortality rate is the percentage of patients for whom the event was observed within the course of the study. The 95% confidence interval, illustrated by dark shading on the graphs, was obtained by using 5000 bootstrap samples.

### Embodiments

The following list of embodiments is intended to complement, rather than displace or supersede, the previous descriptions.
Embodiment 1. A method of detecting proteins in a biological sample obtained from a patient with ovarian cancer, said method comprising:
   determining the level of at least three proteins in the biological sample, wherein the at least three proteins are selected from ANG-2, HE4, PROSTASIN, EGFR and IL8.
Embodiment 2. The method of embodiment 1, wherein the ovarian cancer is a non-mucinous epithelial ovarian cancer.
Embodiment 3. The method of embodiment 1 or 2, wherein the biological sample is serum, plasma or ascites.
Embodiment 4. The method of any preceding embodiment, wherein the level of the at least three proteins is determined using an immunoassay.
Embodiment 5. The method of embodiment 4, wherein the immunoassay is an electrochemiluminescent assay.
Embodiment 6. The method of any preceding embodiment, wherein the at least three proteins consist of EGFR, HE4 and IL8.
Embodiment 7. The method of any one of embodiments 1 to 5 wherein said determining step comprises determining the level of ANG-2, HE4, PROSTASIN, EGFR and IL8.
Embodiment 8. A method of calculating a quantitative score for a patient with ovarian cancer, comprising:
   determining a level of at least three proteins in a biological sample obtained from the patient, wherein the at least three proteins are selected from ANG-2, HE4, PROSTASIN, EGFR and IL8; and
   calculating a quantitative score for the patient by weighting the level of the at least three proteins by their contribution to a clinical outcome.
Embodiment 9. The method of embodiment 8, wherein the ovarian cancer is a non-mucinous epithelial ovarian cancer.
Embodiment 10. The method of embodiment 8 or 9, wherein the biological sample is serum, plasma or ascites.
Embodiment 11. The method of any one of embodiments 8 to 10, wherein the level of the at least three proteins is determined using an immunoassay.
Embodiment 12. The method of embodiment 11, wherein the immunoassay is an electrochemiluminescent assay.
Embodiment 13. The method of any one of embodiments 8 to 12, wherein the levels of ANG-2, HE4, PROSTASIN, EGFR and IL8 are determined.
Embodiment 14. The method of embodiment 13, wherein the quantitative score is calculated based on the algorithm: hOS(t)=hOOS(t) exp(˜A*ANG2+˜B*HEF+˜C*PROSTASIN−˜D*EGFR+˜E*IL8), wherein h_{OS}(t) is the hazard at time (t) and h0_{OS}(t) is the baseline hazard with overall survival as the outcome, wherein the gene symbols in the equation represent the protein levels, and wherein the coefficients A, B, C, D, and E are the coefficients derived for each respective protein.
Embodiment 15. The method of embodiment 14, wherein the quantitative score is calculated based on the algorithm: h_{OS}(t)=h0_{OS}(t) exp(1.213 ANG2+0.171 HE4+0.102 PROSTASIN−1.406 EGFR+0.207 IL8), wherein h_{OS}(t) is the hazard at time (t) and h0_{OS}(t) is the baseline hazard with overall survival as the outcome, and wherein the gene symbols in the equation represent the protein levels.
Embodiment 16. The method of embodiment 13, wherein the quantitative score is calculated based on the algorithm: h_{PFS}(t)=hO_{PFS}(t) exp(˜A*ANG2+˜B*HEF+˜C*PROSTASIN−˜D*EGFR+˜E*IL8), wherein h_{PFS}(t) is the hazard at time (t) and h0_{PFS}(t) is the baseline hazard with progression free survival as the outcome, wherein the gene symbols in the equation represent the protein levels, and wherein the coefficients A, B, C, D, and E are the coefficients derived for each respective protein.
Embodiment 17. The method of embodiment 16, wherein the quantitative score is calculated based on the algorithm: h_{PFS}(t)=h0_{PFS}(t) exp(0.077 ANG2+0.123 HE4+0.008 PROSTASIN−0.545 EGFR+0.156 IL8), wherein h_{PFS}(t) is the hazard at time (t) and h0_{PFS}(t) is the baseline hazard with progression free survival as the outcome, and wherein the gene symbols in the equation represent the protein levels.
Embodiment 18. The method of any one of embodiments 8 to 12, wherein the levels of EGFR, HE4 and IL8 are determined.
Embodiment 19. The method of embodiment 18, wherein the quantitative score is calculated based on the algorithm: h_{OS}(t)=h0_{OS}(t) exp(˜A*HE4−˜B*EGFR+˜C*IL8), wherein h_{OS}(t) is the hazard at time (t) and h0_{OS}(t) is the baseline hazard with overall survival as the outcome, wherein the gene symbols in the equation represent the protein levels, and wherein the coefficients A, B and C are the coefficients derived for each respective protein.
Embodiment 20. The method of embodiment 19, wherein the quantitative score is calculated based on the algorithm: h_{OS}(t)=h0_{OS}(t) exp(0.234 HE4−1.464 EGFR+0.273 IL8), wherein h_{OS}(t) is the hazard at time (t) and h0_{OS}(t) is the baseline hazard with overall survival as the outcome, and wherein the gene symbols in the equation represent the protein levels.
Embodiment 21. The method of embodiment 18, wherein the quantitative score is calculate based on the algorithm: h_{PFS}(t)=h0_{PFS}(t) exp(˜A*HE4−˜B*EGFR+˜C*IL8), wherein h_{PFS}(t) is the hazard at time (t) and h0_{PFS}(t) is the baseline hazard with progression free survival as the outcome, wherein the gene symbols in the equation represent the protein levels, and wherein the coefficients A, B and C are the coefficients derived for each respective protein.
Embodiment 22. The method of embodiment 21, wherein the quantitative score is calculated based on the algorithm: h_{PFS}(t)=h0_{PFS}(t) exp(0.124 HE4−0.538 EGFR+0.161 IL8), wherein h_{PFS}(t) is the hazard at time (t) and h0_{PFS}(t) is the baseline hazard with progression free survival as the outcome, and wherein the gene symbols in the equation represent the protein levels.
Embodiment 23. A method of predicting a likelihood of a clinical outcome in a patient with ovarian cancer, comprising:
   determining a level of at least three proteins in a biological sample obtained from the patient, wherein the at least three proteins are selected from ANG-2, HE4, PROSTASIN, EGFR and IL8;
   calculating a quantitative score for the patient by weighting the level of the at least three proteins by their contribution to a clinical outcome; and
   predicting a likelihood of a clinical outcome for the patient based on the quantitative score.
Embodiment 24. The method of embodiment 23, wherein an increase in the quantitative score correlates with a decreased likelihood of a positive clinical outcome, and wherein a decrease in the quantitative score correlates with an increased likelihood of a positive clinical outcome.
Embodiment 25. The method of embodiment 23 or 24, wherein a likelihood of a negative clinical outcome for the patient informs a decision to discontinue current ovarian cancer therapy and/or initiate an ovarian cancer therapy, and wherein a likelihood of a positive clinical outcome for the patient informs a decision to monitor the progression of the ovarian cancer and/or continue current ovarian cancer therapy.
Embodiment 26. The method of any one of embodiments 23 to 25, wherein the positive clinical outcome is increased overall survival time.
Embodiment 27. The method of any one of embodiment 23 to 26, wherein the positive clinical outcome is progression free survival.
Embodiment 28. The method of any one of embodiments 23 to 27, wherein the ovarian cancer is a non-mucinous epithelial ovarian cancer.
Embodiment 29. The method of any one of embodiments 23 to 28, wherein the likelihood of a clinical outcome is predicted when the ovarian cancer is first diagnosed.
Embodiment 30. The method of any one of embodiments 23 to 28, wherein the likelihood of a clinical outcome is predicted when the ovarian cancer relapses for the first time 6 to 24 months after an initial treatment.
Embodiment 31. The method of any one of embodiments 23 to 28, wherein the likelihood of a clinical outcome is predicted when the ovarian cancer relapses at any time after an initial treatment.
Embodiment 32. The method of any one of embodiments 23 to 28, wherein the likelihood of a clinical outcome is predicted at any time after a first diagnosis.
Embodiment 33. The method of any one of embodiments 23 to 32, wherein the initial treatment comprises surgery and/or chemotherapy.
Embodiment 34. The method of any one of embodiments 23 to 33, wherein the biological sample is serum, plasma or ascites.
Embodiment 35. The method of any one of embodiments 23 to 34, wherein the level of the at least three proteins is determined using an immunoassay.
Embodiment 36. The method of embodiment 35, wherein the immunoassay is an electrochemiluminescent assay.
Embodiment 37. The method of any one of embodiments 23 to 36, wherein the levels of ANG-2, HE4, PROSTASIN, EGFR and IL8 are determined.
Embodiment 38. The method of embodiment 37, wherein the quantitative score is calculated based on the algorithm: h_{OS}(t)=h0_{OS}(t) exp(˜A*HE4−˜B*EGFR+˜C*IL8), wherein h_{OS}(t) is the hazard at time (t) and h0_{OS}(t) is the baseline hazard with overall survival as the outcome, wherein the gene symbols in the equation represent the protein levels, and wherein the coefficients A, B and C are the coefficients derived for each respective protein.
Embodiment 39. The method of embodiment 38, wherein the quantitative score is calculated based on the algorithm: h_{OS}(t)=h0_{OS}(t) exp(1.213 ANG2+0.171 HE4+0.102 PROSTASIN−1.406 EGFR+0.207 IL8), wherein h_{OS}(t) is the hazard at time (t) and h0_{OS}(t) is the baseline hazard with overall survival as the outcome, and wherein the gene symbols in the equation represent the protein levels.
Embodiment 40. The method of embodiment 37, wherein the quantitative score is calculated based on the algorithm: h_{PFS}(t)=hO_{PFS}(t) exp(˜A*ANG2+˜B*HEF+˜C*PROSTASIN−˜D*EGFR+˜E*IL8), wherein h_{PFS}(t) is the hazard at time (t) and h0_{PFS}(t) is the baseline hazard with progression free survival as the outcome, wherein the gene symbols in the equation represent the protein levels, and wherein the coefficients A, B, C, D, and E are the coefficients derived for each respective protein.
Embodiment 41. The method of embodiment 40, wherein the quantitative score is calculated based on the algorithm: h_{PFS}(t)=h0_{PFS}(t) exp(0.077 ANG2+0.123 HE4+0.008 PROSTASIN−0.545 EGFR+0.156 IL8), wherein h_{PFS}(t) is the hazard at time (t) and h0_{PFS}(t) is the baseline hazard with progression free survival as the outcome, and wherein the gene symbols in the equation represent the protein levels.
Embodiment 42. The method of any one of embodiments 23 to 36, wherein the levels of EGFR, HE4 and IL8 are determined.
Embodiment 43. The method of embodiment 42, wherein the quantitative score is calculated based on the algorithm: h_{OS}(t)=h0_{OS}(t) exp(˜A*HE4−˜B*EGFR+˜C*IL8), wherein h_{OS}(t) is the hazard at time (t) and h0_{OS}(t) is the baseline hazard with overall survival as the outcome, wherein the gene symbols in the equation represent the protein levels, and wherein the coefficients A, B and C are the coefficients derived for each respective protein.
Embodiment 44. The method of embodiment 43, wherein the quantitative score is calculated based on the algorithm: h_{OS}(t)=h0_{OS}(t) exp(0.234 HE4−1.464 EGFR+0.273 IL8), wherein h_{OS}(t) is the hazard at time (t) and h0_{OS}(t) is the baseline hazard with overall survival as the outcome, and wherein the gene symbols in the equation represent the protein levels.
Embodiment 45. The method of embodiment 42, wherein the quantitative score is calculate based on the algorithm: h_{PFS}(t)=h0_{PFS}(t) exp(˜A*HE4−˜B*EGFR+˜C*IL8), wherein h_{PFS}(t) is the hazard at time (t) and h0_{PFS}(t) is the baseline hazard with progression free survival as the outcome, wherein the gene symbols in the equation represent the protein levels, and wherein the coefficients A, B and C are the coefficients derived for each respective protein.
Embodiment 46. The method of embodiment 45, wherein the quantitative score is calculated based on the algorithm: h_{PFS}(t)=h0_{PFS}(t) exp(0.124 HE4−0.538 EGFR+0.161 IL8), wherein h_{PFS}(t) is the hazard at time (t) and h0_{PFS}(t) is the baseline hazard with progression free survival as the outcome, and wherein the gene symbols in the equation represent the protein levels.
Embodiment 47. A set of reagents to measure the levels of three or more biomarkers in a patient with ovarian cancer, wherein the biomarkers comprise ANG2, EGFR, HE4, IL8 and PROSTASIN and their measurable fragments.
Embodiment 48. The set of reagents of embodiment 47, wherein the reagents are binding molecules.
Embodiment 49. The set of reagents of embodiment 48, wherein the binding molecules are antibodies.
Embodiment 50. A test kit comprising the set of reagents of embodiment 47.
Embodiment 51. The test kit of embodiment 50, further comprising written instructions for performing an evaluation of biomarkers to predict the likelihood of ovarian cancer in a subject.

1-22. (canceled)

23. A method of predicting a likelihood of a clinical outcome in a patient with ovarian cancer, comprising:
determining a level of at least three proteins in a biological sample obtained from the patient, wherein the at least three proteins are selected from ANG-2, HE4, PROSTASIN, EGFR and IL8;
calculating a quantitative score for the patient by weighting the level of the at least three proteins by their contribution to a clinical outcome; and
predicting a likelihood of a clinical outcome for the patient based on the quantitative score.

24. The method of claim 23, wherein an increase in the quantitative score correlates with a decreased likelihood of a positive clinical outcome, and wherein a decrease in the quantitative score correlates with an increased likelihood of a positive clinical outcome.

25. The method of claim 23, wherein a likelihood of a negative clinical outcome for the patient informs a decision to discontinue current ovarian cancer therapy and/or initiate an ovarian cancer therapy, and wherein a likelihood of a positive clinical outcome for the patient informs a decision to monitor the progression of the ovarian cancer and/or continue current ovarian cancer therapy.

26. The method claim 24, wherein the positive clinical outcome is increased overall survival time.

27. The method of claim 24, wherein the positive clinical outcome is progression free survival.

28. The method of claim 23, wherein the ovarian cancer is a non-mucinous epithelial ovarian cancer.

29. The method of claim 23, wherein the likelihood of a clinical outcome is predicted when the ovarian cancer is first diagnosed, when the ovarian cancer relapses for the first time 6 to 24 months after an initial treatment, when the ovarian cancer relapses at any time after an initial treatment, or at any time after a first diagnosis.

30. (canceled)

31. (canceled)

32. (canceled)

33. The method of claim 29, wherein the initial treatment comprises surgery and/or chemotherapy.

34. The method of claim 23, wherein the biological sample is serum, plasma or ascites.

35. The method of claim 23, wherein the level of the at least three proteins is determined using an immunoassay.

36. The method of claim 35, wherein the immunoassay is an electrochemiluminescent assay.

37. The method of claim 23, wherein the levels of ANG-2, HE4, PROSTASIN, EGFR and IL8 are determined.

38. The method of claim 37, wherein the quantitative score is calculated based on the algorithm: h_{OS}(t)=h0_{OS}(t) exp(˜A*HE4−˜B*EGFR+˜C*IL8), wherein h_{OS}(t) is the hazard at time (t) and h0_{OS}(t) is the baseline hazard with overall survival as the outcome, wherein the gene symbols in the equation represent the protein levels, and wherein the coefficients A, B and C are the coefficients derived for each respective protein.

39. The method of claim 38, wherein the quantitative score is calculated based on the algorithm: h_{OS}(t)=h0_{OS}(t) exp(1.213 ANG2+0.171 HE4+0.102 PROSTASIN−1.406 EGFR+0.207 IL8), wherein h_{OS}(t) is the hazard at time (t) and h0_{OS}(t) is the baseline hazard with overall survival as the outcome, and wherein the gene symbols in the equation represent the protein levels.

40. The method of claim 37, wherein the quantitative score is calculated based on the algorithm: h_{PFS}(t)=hO_{PFS}(t) exp(˜A*ANG2+˜B*HEF+˜C*PROSTASIN−˜D*EGFR+˜E*IL8), wherein h_{PFS}(t) is the hazard at time (t) and h0_{PFS}(t) is the baseline hazard with progression free survival as the outcome, wherein the gene symbols in the equation represent the protein levels, and wherein the coefficients A, B, C, D, and E are the coefficients derived for each respective protein

41. The method of claim 40, wherein the quantitative score is calculated based on the algorithm: h_{PFS}(t)=h0_{PFS}(t) exp(0.077 ANG2+0.123 HE4+0.008 PROSTASIN−0.545 EGFR+0.156 IL8), wherein h_{PFS}(t) is the hazard at time (t) and h0_{PFS}(t) is the baseline hazard with progression free survival as the outcome, and wherein the gene symbols in the equation represent the protein levels.

42. The method of claim 23, wherein the levels of EGFR, HE4 and IL8 are determined.

43. The method of claim 42, wherein the quantitative score is calculated based on the algorithm: h_{OS}(t)=h0_{OS}(t) exp(˜A*HE4−˜B*EGFR+˜C*IL8), wherein h_{OS}(t) is the hazard at time (t) and h0_{OS}(t) is the baseline hazard with overall survival as the outcome, wherein the gene symbols in the equation represent the protein levels, and wherein the coefficients A, B and C are the coefficients derived for each respective protein.

44. The method of claim 43, wherein the quantitative score is calculated based on the algorithm: h_{OS}(t)=h0_{OS}(t) exp(0.234 HE4−1.464 EGFR+0.273 IL8), wherein h_{OS}(t) is the hazard at time (t) and h0_{OS}(t) is the baseline hazard with overall survival as the outcome, and wherein the gene symbols in the equation represent the protein levels.

45. The method of claim 42, wherein the quantitative score is calculate based on the algorithm: h_{PFS}(t)=h0_{PFS}(t) exp(˜A*HE4−˜B*EGFR+˜C*IL8), wherein h_{PFS}(t) is the hazard at time (t) and h0_{PFS}(t) is the baseline hazard with progression free survival as the outcome, wherein the gene symbols in the equation represent the protein levels, and wherein the coefficients A, B and C are the coefficients derived for each respective protein.

46. The method of claim 45, wherein the quantitative score is calculated based on the algorithm: h_{PFS}(t)=h0_{PFS}(t) exp(0.124 HE4−0.538 EGFR+0.161 IL8), wherein h_{PFS}(t) is the hazard at time (t) and h0_{PFS}(t) is the baseline hazard with progression free survival as the outcome, and wherein the gene symbols in the equation represent the protein levels.

47. A set of reagents to measure the levels of three or more biomarkers in a patient with ovarian cancer, wherein the biomarkers comprise ANG2, EGFR, HE4, IL8 and PROSTASIN and their measurable fragments.

48. The set of reagents of claim 47, wherein the reagents are binding molecules.

49. The set of reagents of claim 48, wherein the binding molecules are antibodies.

50. A test kit comprising the set of reagents of claim 47.

51. The test kit of claim 50, further comprising written instructions for performing an evaluation of biomarkers to predict the likelihood of ovarian cancer in a subject.
